(19) United States
(12) Patent Application Publication
(10) Pub. No.: US 2025/0163013 A1
(43) Pub. Date: May 22, 2025

(54) TREATMENT OF GENITOURINARY SYNDROME OF MENOPAUSE

(71) Applicant: THE REGENTS OF THE UNIVERSITY OF COLORADO, a body corporate, Denver, CO (US)

(72) Inventors: Steven NORDEEN, Westminster, CO (US); Michael WEMPE, Richmond, KY (US); Vijay KUMAR, Aurora, CO (US)

(21) Appl. No.: 18/833,240

(22) PCT Filed: Jan. 24, 2023
(86) PCT No.: PCT/US23/61130

§ 371 Date: Jul. 25, 2024

Related U.S. Application Data
(60) Provisional application No. 63/302,668, filed on Jan. 25, 2022.

(51) Int. Cl. C07D 311/32; A61K 31/352; A61P 15/02

(52) U.S. Cl. CPC C07D 311/32; A61K 31/352; A61P 15/02

(57) ABSTRACT

Compounds and compositions useful in methods of treating genitourinary syndrome of menopause (GSM). These compounds, and compositions containing these compounds, may be used as an alternative to systemic hormonal therapies or to localized therapies using estradiol or other forms of steroidal estrogens.

### TECHNICAL FIELD

This invention relates generally to the field of pharmacology and medicine. In particular, the invention relates to compounds for treating the genitourinary syndrome of menopause (GSM) in an individual in need thereof.

### BACKGROUND

The genitourinary syndrome of menopause (GSM) is a relatively new term, first introduced in 2014 by a consensus of the International Society for the Study of Women's Sexual Health and the North American Menopause Society. GSM, previously known (and still often diagnosed) as vulvovaginal atrophy, atrophic vaginitis, and/or urogenital atrophy, is a term that describes the spectrum of changes caused by the reduction or lack of estrogens during menopause. While the majority of women suffering from GSM are of older age, with 50-70% of postmenopausal women symptomatic to some degree, GSM-like symptoms may also be present in about 15% of premenopausal women due to a hypoestrogenic state. GSM remains extremely underdiagnosed despite its high prevalence, mostly because of the reluctance among women to seek help due to embarrassment, or due to a tendency among many women to consider it a normal feature of aging. But the reluctance of healthcare professionals to address these issues constitutes the major cause of the lack of awareness about this syndrome among affected women.

Clinically, GSM is a chronic, progressive condition of the vulvovaginal and lower urinary tract characterized by a broad spectrum of symptoms including vaginal dryness (leading to vaginal irritation, burning, and itching), leukorrhea, thinning/graying pubic hair, vaginal/pelvic pain and pressure, vaginal vault prolapse, dyspareunia, reduced vaginal lubrication, post-coital bleeding, decreased sexual arousal, orgasm, and/or desire, loss of libido, dysorgasmia, dysuria, urinary urgency/stress, incontinence, recurrent urinary tract infections, urethral prolapse, and/or ischemia of vesical trigone.

Most of the symptoms of GSM are attributed to the lack of estrogen that characterizes menopause. There are three types of endogenous estrogens (estradiol, estrone, and estriol) with varying levels that change with age. Estradiol prevails in premenopausal women, whereas estrone is the predominant estrogen in menopause. Estrogen receptors (a and b) are present in the vagina, the vestibule of the vulva, urethra, trigone of the bladder, and on autonomic and sensory neurons in the vagina and vulva. In postmenopausal women, only estrogen receptor a (alpha) is present in the vaginal epithelium and estrogen receptor b (beta) gene expression is either extremely low or not found. Irritation and trauma occurring during sexual intercourse is the result of the hypoestrogenic environment of urogenital tract tissue and reflects changes in the thickness of the vaginal epithelium and lamina propria, atrophy of smooth muscles, reduction of vaginal-area blood flow, and loss of tissue elasticity (decreased concentration of collagen, elastin, and hyaluronic acid). Diminution of glycogen concentration in vaginal cells results in an alteration in the vaginal microbiome (fewer lactobacilli) and an increase of vaginal pH. Increase in vaginal pH following loss of estrogen is also due to fewer mature cells sloughed from the surface epithelium. These sloughed, dead cells are a primary source of glycogen that the microbiome metabolizes to lactic acid to support a low pH and a normal, healthy microbiome. The combined effect of these pathophysiological changes results in changes of the urogenital system, mainly as alterations of the vaginal mucosa (pallor and fragility) and discharge, changes in vaginal microbe consistency, a decrease of pubic hair and subcutaneous fat of labia majora, and loss of size of labia minora and vestibular bulbs. In the lower urinary tract, these changes affect the capacity and contractile ability of the bladder, the urethral sphincter, and the functionality of pelvic floor muscles.

The primary goal of GSM's treatment is symptom relief. The available treatment options include lifestyle changes (maintenance of sexual activity, smoking cessation), vaginal lubricants and moisturizers, liquid lidocaine compresses to the vulvar vestibule, microablative fractional CO₂ laser, non-ablative photothermal erbium (YAG laser), oral ospemifene (a selective estrogen receptor modulator (SERM)), phytoestrogens, oral vitamin D, vaginal vitamin E, probiotics, testosterone, and/or dehydroepiandrosterone. But these treatments are considered weak, at best, and more persistent cases are treated with hormonal therapy, with estrogen products generally considered the “gold standard” treatment. Hormonal therapy includes estrogen-based products that can be administered through different routes (vaginally, orally, transdermally or subcutaneously), with vaginal tablets or creams showing the best outcomes. Estrogens delivered orally, in many cases, do not lead to beneficial effects on the lower genital tract. Locally administered, intravaginal estrogen options (tablets, rings, creams) with low estradiol concentration (550 mcg estradiol or 50 mcg estriol) have shown good outcomes in treating GSM symptoms such as dryness, itchiness, vaginal mucosa friability, and dyspareunia. Menopausal hormone replacement therapy (MHT) or hormone replacement therapy (HRT) using estrogen or estradiol often causes nausea and carries the significant increased risk of hormone-sensitive carcinoma, particularly breast and endometrial cancers.

There remains, therefore, a great need for an effective treatment for GSM and its many symptoms that displays only slight or negligible systemic estrogen effects, including the increased risk of breast and endometrial cancers.

### SUMMARY

This disclosure provides methods of treating genitourinary syndrome of menopause in an individual. These methods include the administration of flavone compounds that act locally to activate estrogen receptors, thereby producing estrogenic effects, but are rapidly inactivated in the circulation to prevent any systemic estrogenic effects.

Accordingly, in one aspect, a compound of this disclosure is a flavone compound, or a pharmaceutically acceptable salt thereof, having the chemical structure:

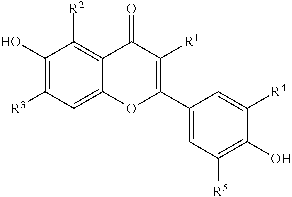

wherein: R¹ is methyl, ethyl, propyl, butyl, O-methyl, O-ethyl, O-propyl, O-butyl, methyl-OH, ethyl-OH, or halogen, and wherein R², R³, R⁴, and R⁵ are each independently hydrogen or halogen. In a related aspect, a compound of this disclosure is a flavone compound, or a pharmaceutically acceptable salt thereof, having a chemical structure selected from the group consisting of the compounds of Table 1:

TABLE 1
Flavone compounds of this disclosure.

| # | COMPOUND STRUCTURE | CHEMICAL NAME |
|---|---|---|
| 1 | 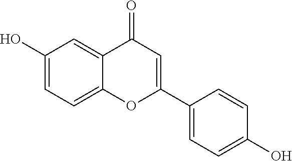 [SMILES: O=c1cc(-c2ccc(O)cc2)oc2ccc(O)cc12] | 6-hydroxy-2-(4-hydroxyphenyl)-4H- chromen-4-one |
| 2 | 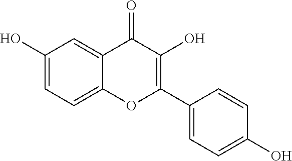 [SMILES: O=c1c(O)c(-c2ccc(O)cc2)oc2ccc(O)cc12] | 3,6-dihydroxy-2-(4-hydroxyphenyl)-4H- chromen-4-one |
| 3 | 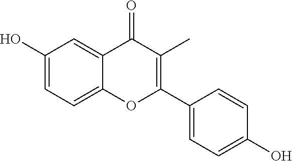 [SMILES: Cc1c(-c2ccc(O)cc2)oc2ccc(O)cc2c1=O] | 6-hydroxy-2-(4-hydroxyphenyl)-3-methyl-4H- chromen-4-one |
| 4 | 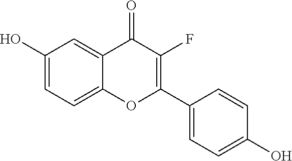 [SMILES: O=c1c(F)c(-c2ccc(O)cc2)oc2ccc(O)cc12] | 3-fluoro-6-hydroxy-2-(4-hydroxyphenyl)-4H- chromen-4-one |
| 5 | 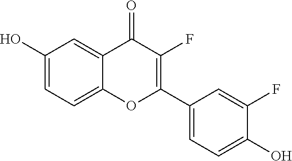 [SMILES: O=c1c(F)c(-c2ccc(O)c(F)c2)oc2ccc(O)cc12] | 3-fluoro-6-hydroxy-2-(3-fluoro-4-hydroxyphenyl)- 4H-chromen-4-one |

In another aspect, this disclosure provides pharmaceutical compositions comprising at least one compound of this disclosure and at least one pharmaceutically acceptable additive. Preferred pharmaceutical compositions of this disclosure are formulated for intravaginal administration or application. Formulations of the pharmaceutical compositions of the invention for intravaginal administration may be presented as a suppository, which may be prepared by mixing one or more compounds of this disclosure with one or more suitable nonirritating excipients or carriers comprising, for example, cocoa butter, polyethylene glycol, a suppository wax or salicylate, and which is solid at room temperature, but liquid at body temperature and, therefore, will melt in the vaginal cavity and release one or more compound(s) of this disclosure. Formulations of the present invention which are suitable for vaginal administration also include pessaries, tampons, creams, gels, pastes, foams, or spray formulations containing such carriers as are known in the art to be appropriate.

Another aspect of this disclosure provides pharmaceutical kits containing a pharmaceutical composition of this disclosure, prescribing information for the composition, and a container.

Another aspect of this disclosure provides methods for stimulating estrogen receptors in an individual, including administering to the subject a therapeutically effective amount of a compound of this disclosure, or a pharmaceutically acceptable salt thereof, or a pharmaceutical composition of this disclosure. In these methods, the individual typically is a human female. In these methods, the individual may have at least one of: vulvovaginal atrophy, vaginal dryness, incontinence, dyspareunia, and recurrent urinary tract infections. For example, the individual may have vulvovaginal atrophy resulting from loss of estrogen during perimenopause and/or menopause, surgical removal of ovaries, chemically induced estrogen suppression (such as estrogen receptor antagonists and aromatase inhibitors) during and after treatment of breast cancer. In these methods, the administration may increase growth and/or differentiation of cells of the vaginal epithelium in the individual's vagina. In these methods, the administration may inhibit growth and/or activity of at least one of: pathogenic bacteria, yeast, and viruses. In these methods, the administration may increase moisture content of the vaginal epithelium in the individual's vagina. In these methods, the administration may improve barrier function in the individual's vaginal epithelium. In these methods, the administration may increase vascularity and blood flow in the individual's vagina.

This Summary is neither intended nor should it be construed as representative of the full extent and scope of the present disclosure. Moreover, references made herein to “the present disclosure” or aspects thereof, should be understood to mean certain embodiments of the present invention and should not necessarily be construed as limiting all embodiments to a particular description. The present invention is set forth in various levels of detail in this Summary as well as in the attached drawings and the Detailed Description and no limitation as to the scope of the present invention is intended by either the inclusion or non-inclusion of elements, components, etc. in this Summary. Additional aspects of the present invention will become more readily apparent from the Detailed Description, particularly when taken together with the figures.

### BRIEF DESCRIPTION OF FIGURES

FIG. 1 depicts a general synthesis scheme for the synthesis of 6-hydroxy-2-(4-hydroxyphenyl)-3-methyl-4H-chromen-4-one (compound 3 of Table 1).

FIG. 2A depicts a general synthesis scheme for the synthesis of 6-hydroxy-2-(4-hydroxyphenyl)-4H-chromen-4-one (compound 1 of Table 1) and 3-fluoro-6-hydroxy-2-(4-hydroxyphenyl)-4H-chromen-4-one (compound 4 of Table 1).

FIG. 2B depicts an alternative synthesis scheme of 3-fluoro-6-hydroxy-2-(4-hydroxyphenyl)-4H-chromen-4-one (compound 4 of Table 1).

FIG. 3 depicts a general synthesis scheme for the synthesis of 3-fluoro-6-hydroxy-2-(3-fluoro-4-hydroxyphenyl)-4H-chromen-4-one (compound 5 of Table 1).

FIG. 4 shows the results of a cell culture-reporter gene assay of estrogenic activity after treatment with flavone test compounds for 20 hours at the indicated doses. Estrogenic activity was compared to an optimal dose of estradiol (1 nM).

FIG. 5A shows representative photomicrographs of hematoxylin and eosin-stained squamous epithelium from vaginal canal following treatment with test compound 4 (3-fluoro-6-hydroxy-2-(4-hydroxyphenyl)-4H-chromen-4-one; abbreviated “3F” in figure), control (vehicle), or estradiol (positive control).

FIG. 5B shows the quantification of an in vivo experiment to assess regeneration of the vaginal lining following treatment with estradiol or two different doses of compound 4. Independent measurement from mid-vagina (sections 3-4) and vagina near the cervix (sections 6-7) are shown. Twenty-five separate measurements were performed on sections 3-4 and also on sections 6-7 for each animal. There were 6 animals per treatment group, therefore each bar represents the mean of 150 measurements with the vertical line indicating one standard deviation.

FIG. 5C is bar graphs showing the mean cornification index values of the vaginal tissues (sections 3-4, left, or sections 6-7 right) from the rats treated with vehicle (ctrl), estradiol, or compound 4 (“3F”) of this disclosure.

FIG. 5D shows a photomicrograph of vaginal tissue with immunohistochemical staining of Ki-67 protein from rats treated with compound 4 of this disclosure. Bracket indicates basal and parabasal layers of the tissue.

FIG. 5E is a bar graph showing the mean number of Ki-67 positive cells in vaginal tissue samples from rats treated with vehicle (ctrl), estradiol, or compound 4 (“3F”) of this disclosure.

FIG. 6A shows photomicrographs of uterine and bladder tissue from rats treated with compounds of this disclosure. Bracket E indicates uterine endometrium and bracket M indicates myometrium. Bracket B indicates the urothelium of the bladder.

FIG. 6B are bar graphs showing the group mean of thickness of endometrium, myometrium, and urothelium tissues from rats treated with vehicle (ctrl), estradiol, or two different levels of compound 4 (“3F”) of this disclosure.

FIG. 6C is a photomicrograph of uterine tissue with immunohistochemical (IHC) staining of Ki-67 protein from rats treated with compound 4 (“3F”) of this disclosure.

FIG. 6D is a bar graph showing the mean percentage Ki-67 positive uterine cells in tissue samples from rats treated with vehicle (ctrl), estradiol, or compound 4 (“3F”) of this disclosure.

FIG. 7A shows photomicrographs of vaginal tissue samples from rats treated with vehicle, estradiol, or compound 4 (“3F”) of this disclosure with immunohistochemical staining for lactoferrin.

FIG. 7B is a bar graph showing the group mean scores of a semi-quantitative scoring system for lactoferrin expression in vaginal epithelium tissues (represented by immunohistochemical staining for lactoferrin) from rats treated with vehicle, estradiol, or compound 4 (“3F”) of this disclosure.

FIG. 8A shows photomicrographs of vaginal tissue samples from rats treated with vehicle, estradiol, or compound 4 (“3F”) of this disclosure stained with Alcian Blue-Periodic acid-Schiff (AB-PAS).

FIG. 8B is a bar graph showing the mean scores of a semi-quantitative scoring system for mucous production in vaginal epithelium tissues (represented by AB-PAS staining) from rats treated with vehicle (ctrl), estradiol, or compound 4 (“3F”) of this disclosure.

FIG. 8C shows photomicrographs of vaginal tissue samples from rats treated with vehicle, estradiol, or compound 4 (“3F”) of this disclosure with immunohistochemical staining for mucin 5B, acidic mucin.

FIG. 8D is a bar graph showing the group mean scores of a semi-quantitative scoring system for mucous production in vaginal epithelium tissues (represented by immunohistochemical staining for mucin 5B) from rats treated with vehicle, estradiol, or compound 4 (“3F”) of this disclosure.

FIG. 9 is a graph depicting the effect on growth of estrogen-responsive MCF-7EV cells in tissue culture after treatment with escalating doses of compounds 4 and 5 (“3F” and “33′DiF” respectively) of this disclosure compared to an optimal dose of estradiol (“E2”).

### DETAILED DESCRIPTION

As used herein, the singular forms “a”, “an”, and “the” include plural reference unless the context clearly dictates otherwise. For example, reference to “a cell” includes a plurality of such cells and equivalents thereof known to those skilled in the art, and so forth. Additionally, the terms “comprising”, “including”, and “containing” can be used interchangeably.

Whenever a range of values is given in the specification, for example, a temperature range, a time range, or a composition or concentration range, all intermediate ranges and subranges, as well as all individual values included in the ranges given are intended to be included in the disclosure. As used herein, ranges specifically include the values provided as endpoint values of the range. For example, a range of 1 to 100 specifically includes the end point values of 1 and 100. Any subranges or individual values in a range or subrange that are included in this description can also be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention described herein suitably may be practiced in the absence of any element or elements, limitation or limitations which is not specifically disclosed herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, or operations. Also, the term “or” is used in its inclusive sense (and not in its exclusive sense) so that when used, for example, to connect a list of elements, the term “or” means one, some, or all of the elements in the list.

All references cited throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (a reference that is partially inconsistent is incorporated by reference except for the partially inconsistent portion of the reference).

As used herein, a “therapeutically effective amount” refers to an amount of formulation, pharmaceutical composition, or reagent in a pharmaceutically acceptable carrier or a physiologically acceptable salt of an active compound that is of sufficient quantity to ameliorate the undesirable state of the patient, animal, material, or object so treated. “Ameliorate” refers to a lessening of the detrimental effect of the GSM disease state or disorder, or reduction in contamination, in the receiver of the treatment. The therapeutically active compounds of this disclosure are effective over a wide dosage range and are generally administered in a therapeutically effective amount. The dosage and manner of administration will be defined by the application of the compound(s) and can be determined by routine methods of clinical testing to find the optimum dose. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

“Pharmaceutical agent or drug” as used herein, refers to a chemical compound or pharmaceutical composition capable of inducing a desired therapeutic or prophylactic effect when properly administered to an individual.

“Pharmaceutically acceptable carrier” as used herein, refers to conventional pharmaceutical carriers useful in the methods disclosed herein. Remington's Pharmaceutical Sciences, by E. W. Martin, Mack Publishing Co., Easton, Pa., 15th Edition (1975), describes compositions and formulations suitable for pharmaceutical delivery of flavones and additional pharmaceutical agents. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid pharmaceutical compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, Tweens, sodium acetate, or sorbitan monolaurate.

The terms “pharmaceutically acceptable salt” or “pharmaceutically acceptable ester” refers to salts or esters prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid, and the like.

For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

The pharmaceutically acceptable acid and base addition salts as mentioned above are meant to comprise the therapeutically active non-toxic acid and base addition salt forms which the compounds can form. The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic, and like acids. Conversely, these salt forms can be converted into the free base form by treatment with an appropriate base.

The compounds containing an acidic proton may also be converted into their non-toxic metal or amine addition salt forms by treatment with appropriate organic and inorganic bases. Appropriate base salt forms comprise, for example, the ammonium salts, the alkali and earth alkaline metal salts, e.g., the lithium, sodium, potassium, magnesium, calcium salts and the like, salts with organic bases, e.g., the benzathine, N-methyl-D-glucamine, hydrabamine salts, and salts with amino acids such as, for example, arginine, lysine, and the like.

When a group of substituents is disclosed herein, it is understood that all individual members of that group and all subgroups, including any isomers, enantiomers, and diastereomers of the group members, are disclosed separately.

When a Markush group or other grouping is used herein, all individual members of the group and all combinations and subcombinations possible of the group are intended to be individually included in the disclosure. When a compound is described herein such that a particular isomer, enantiomer, or diastereomer of the compound is not specified, for example, in a formula or in a chemical name, that description is intended to include each isomer and enantiomer of the compound described individually, or in any combination. Additionally, unless otherwise specified, all isotopic variants of compounds disclosed herein are intended to be encompassed by the disclosure. As a brief illustration, it will be understood that any one or more hydrogens in a molecule disclosed can be replaced with deuterium or tritium.

Isotopic variants of a molecule are generally useful as standards in assays for the molecule and in chemical and biological research related to the molecule or its use. Methods for making such isotopic variants are known in the art.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this disclosure.

The terms “to modulate” and “modulates” means to increase or decrease. These terms can refer to increasing or decreasing an activity, level, or function of a molecule (e.g., protein, peptide, nucleic acid, small molecule, metabolite), or effecting a change with respect to one or more biological or physiological mechanisms, effects, responses, functions, pathways, or activities.

The terms “agent” and “therapeutic agent” as used herein refer to a chemical entity or biological product, or combination of chemical entities or biological products, administered to a subject (a mammal such as a human) to treat a disease or condition (e.g., GSM). Examples of therapeutic agents include the compounds of this disclosure.

The terms “patient,” “subject” and “individual” are used interchangeably herein, and mean a subject, typically a mammal, to be treated, diagnosed, and/or to obtain a biological sample from. Subjects include, but are not limited to, humans, non-human primates, horses, cows, sheep, pigs, rats, mice, insects, dogs, and cats. A human female in need of treatment for the GSM is an example of a subject.

The terms “sample,” “patient sample,” “biological sample,” and the like, encompass a variety of sample types obtained from a patient, individual, or subject and can be used in a therapeutic drug screening, diagnostic or monitoring assay. The patient sample may be obtained from a healthy subject, a diseased patient or a patient having associated symptoms of a particular disease or disorder (e.g., the GSM). The definition specifically encompasses blood and other liquid samples of biological origin (including, e.g., vaginal fluid, plasma, serum, peripheral blood), solid tissue samples such as a biopsy specimen or tissue cultures or cells derived therefrom and the progeny thereof. These terms encompass a clinical sample, and also include cells in culture, cell supernatants, tissue samples, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

As used herein, the terms “therapeutic treatment” and “therapy” are defined as the application or administration of a therapeutic agent (e.g., a compound of this disclosure) or therapeutic agents to a patient who has a disease, a symptom of disease or a predisposition toward a disease, with the purpose to cure, heal, alleviate, relieve, alter, remedy, ameliorate, improve, or affect the disease, the symptoms of disease, or the predisposition toward disease.

This disclosure provides methods of alleviating or treating the genitourinary syndrome of menopause (GSM) in an individual (e.g., a human female having GSM). These methods include administering a compound of this disclosure, as described above, to an individual having GSM. In these methods, administration of a compound of this disclosure results in a reduction or elimination of one or more symptoms of GSM. In these methods, the administration of an effective amount of a compound of this disclosure may be administered to an individual in the absence of any hormones (e.g., estrogen or estradiol). In these methods, the administration of an effective amount of a compound of this disclosure may effectively reduce or eliminate vaginal dryness, desquamation, and/or enhance vaginal tissue repair, or vaginal blood flow.

These methods of treating GSM in an individual include administering to the individual (e.g., a human female) a compound of this disclosure, or a pharmaceutical composition including a pharmaceutically acceptable carrier and a therapeutically effective amount of a compound of this disclosure.

Methods of synthesizing compounds of this disclosure are described in detail, below, and are generally known in the arts of flavone synthesis and medicinal chemistry. Compounds of this disclosure may be synthesized or prepared according to any suitable method, or may be commercially obtained, in order to practice the methods of this disclosure.

### Pharmaceutical Compositions

The compounds of this disclosure may be formulated with any pharmaceutically acceptable carrier according to conventional pharmaceutical practice (see, e.g., Remington: The Science and Practice of Pharmacy (21st ed.), ed. A. R. Gennaro, Lippincott Williams & Wilkins, (2005) and Encyclopedia of Pharmaceutical Technology, (3rd ed.) eds. J. Swarbrick and J. C. Boylan, Marcel Dekker, CRC Press, New York (2006), a standard text in this field, and in USP/NF). A description of exemplary pharmaceutically acceptable additives, carriers, excipients, and diluents, as well as pharmaceutical formulations, can be found in Remington, supra. Other substances may be added to the compounds and compositions to stabilize and/or preserve them. In preferred formulations, the pharmaceutical compositions of this disclosure are formulated for administration to the organs of the reproductive system and the urinary system (i.e., the urogenital system or genitourinary system).

Pharmaceutical compositions of this disclosure, comprising at least one compound of this disclosure, may be formulated as a cream, lotion, paste, ointment, and similar formats applied by contact to skin, or mucosal tissue with respect to genitourinary administration. In some formulations, the pharmaceutical compositions may be administered to a target area via spray, drops, wash, swab, sponge, absorbent dressing, soaking, submerging, instillation, or irrigation.

Generally, the pharmaceutical compositions described herein are delivered intravaginally inside of a delivery vehicle, for example a capsule, suppository, or intravaginal ring. Such capsules may be soft capsules made of materials well known in the pharmaceutical arts, for example gelatin. However, the delivery vehicle is integral with the pharmaceutical composition (i.e., the pharmaceutical composition is the delivery vehicle). Thus, the formulation of the pharmaceutical composition forming these intravaginal delivery vehicles may be a gel, cream, ointment, tablet, or other preparation that is directly applied and absorbed vaginally.

Ring-shaped vaginal drug delivery devices (“vaginal rings”) are well known in the art. See, for example, U.S. Pat. No. 4,292,965 (Nash et al.) and U.S. Pat. No. 4,822,616 (Zimmermann et al.), U.S. Pat. No. 3,995,633 (Gougeon), U.S. Pat. No. 3,995,634 (Drobish), U.S. Pat. No. 4,237,885 (Wong et al.), and U.S. Pat. No. 5,989,581. Such devices are designed to deliver a relatively constant dose of drug to the vagina, usually over a period of weeks to months. Typically, they are made of a silicone elastomer and contain a drug released by diffusion though the elastomer. Vaginal rings have been developed for delivering steroids to treat post-menopausal vaginal conditions, as well as for contraception and hormone replacement therapy. Women may prefer vaginal rings to oral delivery for several reasons, particularly their convenience, privacy, long-term delivery capacity, and effectiveness. Vaginal rings provide a regulated dose of drug with minimal involvement or attention by the user. They also avoid the first pass of orally administered drugs through the liver. Such vaginal rings are typically composed of a thermoplastic polymer or elastomer material suitable for pharmaceutical use, such as polysiloxanes, polyurethane, polyethylene, ethylene-vinyl acetate copolymers, cellulose, copolymers of polystyrene, polyacrylates and various types of polyamides and polyesters. The ethylene-vinyl acetate copolymer (EVA) is highly preferred due to its excellent mechanical and physical properties (e.g., solubility of the drug in the material). The EVA material can be any commercially available ethylene-vinyl acetate copolymer, such as the products available under the names Elvax®, Evatane®, Lupolen®, Movriton®, Ultrathene® and Vestypar®.

For pharmaceutical compositions of this disclosure formulated for intravaginal delivery, the delivery vehicle is designed for ease of insertion. Accordingly, the delivery vehicle is sized whereby it can be comfortably inserted into the vagina. Such vaginal delivery vehicles may be prepared in a variety of geometries, such as a tear drop, a cone with frustoconical end, a cylinder, a cylinder with larger “cap” portion, or other shapes suitable for and that ease insertion into the vagina. Such vaginal delivery vehicles may be used in connection with an applicator or may be inserted digitally. Because the site of vaginal atrophy is in the proximal region of the vagina (towards the vaginal opening), the pharmaceutical compositions disclosed herein are preferably designed to be inserted in the proximal portion of the vagina. For an intravaginal drug delivery device (“vaginal ring”), the ring may be manufactured in any size as required. In the case of human use, the ring-shaped device typically has an outer diameter from about 40 mm to about 80 mm, and preferably between 50 mm and 60 mm; the cross-sectional diameter is preferably between about 1 mm and about 12 mm, and preferably between 2 and 6 mm.

Pharmaceutical compositions of this disclosure formulated for genitourinary administration may include one or more components selected from the group consisting of hydrolyzed gelatin, sorbitol-sorbitan solution, water, glycerin, titanium dioxide, FD&C Red #40, ethanol, ethyl acetate, propylene glycol, polyvinyl acetate phthalate, isopropyl alcohol, polyethylene glycol, and/or ammonium hydroxide.

The genitourinary delivery vehicle is designed to remain in the vagina until the pharmaceutical compositions are released. Thus, the delivery vehicle may dissolve intravaginally and may be absorbed into the vaginal tissue with the pharmaceutical composition, which minimizes vaginal discharge.

The pharmaceutical compositions of this disclosure that are formulated for genitourinary delivery are designed to maximize favorable characteristics that lead to patient compliance without sacrificing efficacy. Favorable characteristics include, for example, lack of or reduction of irritation relative to other vaginal delivery systems, lack of or reduction in vaginal discharge of the pharmaceutical composition and delivery vehicle, lack of or reduction of pharmaceutical composition or delivery vehicle residue inside the vagina, ease of administration, or improved efficacy of drug product relative to otherwise similar pharmaceutical compositions. Preferably, the pharmaceutical compositions of this disclosure formulated for vaginal delivery do not leave residue inside the vagina. Rather, the pharmaceutical composition and delivery vehicle are substantially absorbed or dispersed without resulting in unabsorbed residue or unpleasant sensations of non-absorbed or non-dispersed drug product.

The pharmaceutical compositions of this disclosure that are formulated for genitourinary delivery are non-irritating or minimize irritation. Patient irritation may include pain, pruritus (itching), soreness, excessive discharge, swelling, or other similar conditions. Non-irritating or reduced irritation pharmaceutical compositions are measured relative to competing vaginal delivery systems, including tablets, creams, or other intravaginal drug delivery forms.

The pharmaceutical compositions of this disclosure that are formulated for genitourinary delivery reduce or eliminate systemic exposure (e.g., blood circulation of flavone compounds), which improves patient safety by minimizing systemic estrogenic activity that may increase the risk of breast or endometrial cancers.

These pharmaceutical compositions may be liquid pharmaceutical compositions such as a liquid encapsulated in a soft gelatin capsule or other soft capsule. The encapsulated liquid may be a liquid at room temperature or at body temperature. For example, a pharmaceutical composition provided herein may be a liquid formulation contained within a soft gel capsule. Gels, hard fats, or other solid forms that are not liquid at room or body temperature are less desirable in embodiments of the pharmaceutical composition that are liquid.

These pharmaceutical compositions include a compound of this disclosure and may include other ingredients, e.g., colorants, antioxidants, preservatives, oils or fatty acid esters, lecithin, mucoadherent agents, gelling agents, dispersing agents, or other ingredients. However, the addition of other ingredients should be in amounts that do not materially change the solubility of the flavone compounds of this disclosure, the pharmacokinetics of the pharmaceutical composition, or the efficacy of the pharmaceutical composition. Other factors that should be considered when adjusting the ingredients of the pharmaceutical composition include the irritation, vaginal discharge, intravaginal residue, and other relevant factors, for example those that would lead to reduced patient compliance.

### Methods of Treating GSM in an Individual

Methods of treating GSM in an individual (e.g., a human female) include administering a therapeutically effective amount of a compound of this disclosure, typically in the form of a pharmaceutical composition including a pharmaceutically acceptable carrier, to the individual. The compounds and pharmaceutical compositions of this disclosure may be used to treat, reduce, or prevent any condition associated with or caused by GSM. Specific examples of conditions associated with or caused by GSM include vaginal atrophy (VVA), recurrent urinary tract infections, dyspareunia, vaginal dryness, vaginal irritation (e.g., pain), vaginal itching, incontinence, loss of vulvovaginal tissue elasticity, loss of vaginal wall thickness, increased vaginal pH, increased risk of sexually transmitted disease, and decreased immune function. In these methods, the individual may have been diagnosed with vulvovaginal atrophy, atrophic vaginitis, and/or urogenital atrophy.

If the individual has VVA, the VVA can be caused by, or result from, any procedure or condition. For example, in some embodiments, the VVA can result from loss of estrogen during perimenopause and/or menopause. In other embodiments, the individual has VVA resulting from surgical removal of ovaries, or from chemically induced estrogen suppression (such as estrogen receptor antagonists or aromatase inhibitors) during treatment of breast cancer.

In these methods, a compound of this disclosure is typically administered in an amount sufficient to result in one or more of the following: increased depth of the vaginal epithelium, increased mucin production in the individual's vagina; increased moisture in the individual's vagina; increased growth and/or differentiation of cells (e.g., keratinocytes) in the vaginal epithelium of the individual; increased keratin fiber synthesis and keratin fiber cross-linking in the individual's vagina; increased vascularization (angiogenesis) and blood flow in the individual's vagina; decreased undesirable bacterial, fungal and viral growth in the individual's vagina; improved barrier function in the individual's vaginal epithelium; and improvement of the vaginal microbiome (e.g., increase desirable microbial growth in the vagina).

A method of modulating (i.e., improving) an individual's vaginal microbiome includes administering a compound of this disclosure to increase glycogen production, promoting Lactobacillus colonization, and decreasing pH in the individual's vagina. In such methods, increasing glycogen production in the individual is beneficial for vaginal health because glycogen promotes Lactobacillus colonization in the vagina which produces lactic acid and lowers the pH which results in an unfavorable environment for the growth of pathogenic bacteria.

In these methods of treating GSM in an individual, the individual may suffer from vaginal thinning and vaginal atrophy. In such an individual, administering a compound of this disclosure may increase epidermal development and/or keratinocyte differentiation in the individual's vagina. Typically, in such an embodiment, administering a compound of this disclosure results in increased epidermal development and/or keratinocyte differentiation and restoration of the vaginal tissue and alleviation or reversal of the vaginal atrophy in the individual.

In these methods of treating GSM in an individual, the individual may suffer from vaginal dryness. In such individual, administering a compound of this disclosure may increase moisture, lubrication, and blood flow in the vagina.

The methods of this disclosure may further include detecting and/or diagnosing the presence of the GSM in the individual prior to administration of a compound of this disclosure. The detection is typically done prior to administering to the individual a compound or pharmaceutical composition of this disclosure. For example, the methods of treating GSM of this disclosure may include a step of obtaining a sample from the individual and analyzing it to determine if the individual suffers from GSM. Methods of detecting GSM in an individual are well known in the art and include: detection of atrophy of labia majora and minora; and detection of mucosa that appear pale, shiny, and dry. As another example, detection of vaginal rugae disappearance and shortening and narrowing of the vagina which results in greater exposure of the urethral meatus may be used to detect GSM. As another example, the vaginal pH may be measured, and a pH greater than pH 4.6 is indicative of VVA. A further example is use of the vaginal maturation index (VMI).

Any suitable methods of administering a compound or pharmaceutical composition of this disclosure to an individual may be used. In these methods, the compounds and pharmaceutical compositions can be administered to an individual by any suitable route, in particular, administration topically (i.e., both skin and mucosal surfaces), and/or directly to a target site (e.g., vaginal administration).

The methods of treating GSM of this disclosure generally include administration of a therapeutically effective amount of a compound or pharmaceutical composition of this disclosure to an individual (e.g., a human female) in need thereof. Such treatment will be suitably administered to individuals suffering from, having, susceptible to, or at risk for a disease, disorder, or symptom thereof (e.g., GSM). Determination of those individuals “at risk” can be made by any objective or subjective determination by a diagnostic test or opinion of a subject or health care provider.

The compounds and pharmaceutical compositions of this disclosure are preferably administered to an individual in need thereof (e.g., human female having the GSM) in an effective amount, that is, an amount capable of producing a desirable result in a treated individual. Desirable results may include one or more of alleviation or reversal of vaginal atrophy, reduced recurrent urinary tract infections, alleviation of dyspareunia, alleviation or reversal of vaginal dryness, alleviation or elimination of vaginal irritation (e.g., pain), alleviation or elimination of vaginal itching, alleviation or elimination of incontinence, restoration of vaginal wall thickness, decreased vaginal pH, decreased risk of sexually transmitted disease, and/or modification/attenuation of immune function. Such a therapeutically effective amount can be determined according to standard methods. Toxicity and therapeutic efficacy of compounds of this disclosure can be determined by standard pharmaceutical procedures. As is well known in the medical and veterinary arts, dosage for any one individual depends on many factors, including the individual's size, body surface area, age, the composition to be administered, time and route of administration, general health, and other drugs being administered concurrently. A delivery dose of compounds of this disclosure is determined based on preclinical efficacy and safety. Those skilled in the art can determine an appropriate time and duration of therapy that includes the administration of a compound or pharmaceutical composition of this disclosure to achieve the desired preventative or ameliorative effects on the individual treated.

A closely related aspect of this disclosure provides a compound of this disclosure, or a pharmaceutical composition of this disclosure, for use in the treatment of genitourinary syndrome of menopause in an individual.

Another closely related aspect of this disclosure provides the use of a compound of this disclosure, or a pharmaceutical composition of this disclosure, in the manufacture of a medicament for the treatment of genitourinary syndrome of menopause in an individual.

This disclosure also provides kits for alleviating or treating GSM in an individual. A typical kit includes a pharmaceutical composition comprising a compound of this disclosure and a pharmaceutically acceptable carrier, and instructions for using the pharmaceutical composition. Kits also typically include a container and packaging. Instructional materials for preparation and use of the kit components are generally included. While the instructional materials typically include written or printed materials, they are not limited to such. Any medium capable of storing such instructions and communicating them to an end user is encompassed by the kits herein. Such media include, but are not limited to, electronic storage media and the like. Such media may include addresses to internet sites that provide such instructional materials.

It is to be appreciated that any feature or aspect described herein can be claimed in combination with any other feature(s) or aspect(s) as described herein, regardless of whether the features come from the same described embodiment.

The invention now being generally described will be more readily understood by reference to the following examples, which are included merely for the purposes of illustration of certain aspects of the invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

### Synthesis of Flavone Compounds of this Disclosure

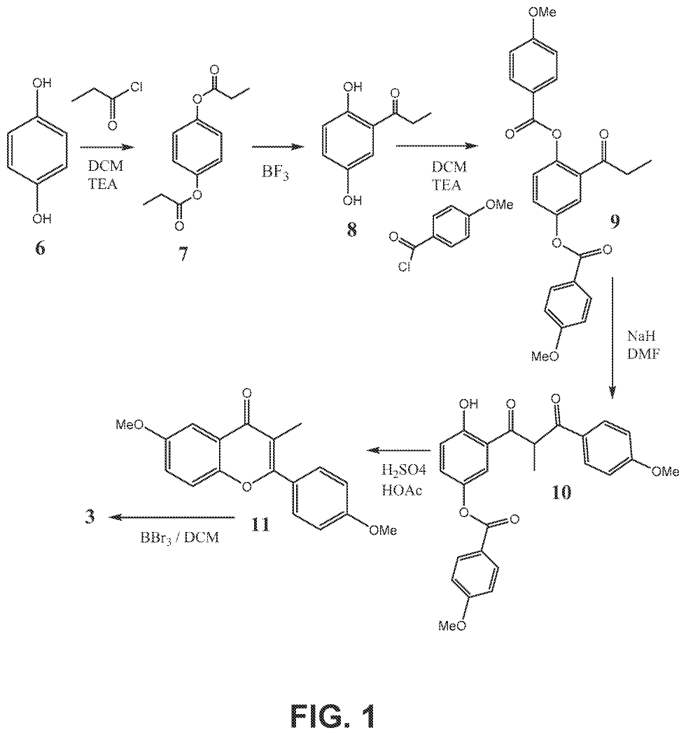

A. Synthesis of 6-hydroxy-2-(4-hydroxyphenyl)-3-methyl-4H-chromen-4-one (compound 3 of Table 1). Following the synthesis scheme depicted in FIG. 1:

1,4-phenylene dipropionate (7): Hydroquinone (6, 1.1 g, 10.0 mmol, 1.0 equiv.) and triethylamine (5.2 mL, 3.7 g, 37.0 mmol, 3.7 equiv.) were dissolved in 150 mL of dry DCM and cooled to 0° C. Propionyl chloride (3.23 mL, 3.42 g, 37.0 mmol, 3.7 equiv.) was then added drop-wise to the solution while stirring. The reaction mixture was allowed to warm to room temperature while stirring overnight. The mixture was then washed three times with 1.0 M NaOH (20 mL) and once with brine (50 mL) and dried over anhydrous sodium sulfate. The material was filtered and the solvent subsequently removed using rotary evaporation under reduced pressure, and the product was purified by silica gel chromatography using 10:1 hexanes/ethyl acetate to give 0.20 g of 7 as a white solid. NMR was consistent.
1-(2,5-dihydroxyphenyl)propan-1-one (8): Boron trifluoride dibutyl etherate (4.0 ml, 19.37 mmol) and (1.0 g, 4.50 mmol) of 1,4-phenylene dipropionate (7) were added to a 100-mL round-bottom flask equipped with a condenser and a stir bar. Heat was then applied, and the mixture was refluxed at 105° C. for 2 h. The resulting solution was allowed to cool to room temperature, and then poured into cold water (100 mL) and extracted with dichloromethane (100 mL). The organic phase was washed again with water (2×50 mL) and dried over anhydrous MgSO₄. The material was filtered and the solvent subsequently removed using rotary evaporation under reduced pressure, and the crude product was purified by silica gel chromatography using 30% acetone/hexane to give 0.40 g (53%) of 8 as a white solid. NMR was consistent.
2-propionyl-1,4-phenylene bis(4-methoxybenzoate) (9): 2,5-Dihydroxypropiophenone (8, 1.7 g, 10.0 mmol, 1.0 equiv.) and triethylamine (5.2 mL, 3.7 g, 37.0 mmol, 3.7 equiv.) were dissolved in 150 mL of dry DCM and cooled to 0° C. 4-Methoxybenzoyl chloride (5.0 mL, 6.31 g, 37.0 mmol, 3.7 equiv.) was then added drop wise to the solution while stirring. The reaction mixture warmed to room temperature while stirring overnight. The mixture was then washed three times with 1.0 M NaOH (50 mL) and once with brine (50 mL) and dried over anhydrous sodium sulfate. The material was filtered and the solvent was subsequently removed using rotary evaporation under reduced pressure and the product was purified by silica gel chromatography using 10:1 hexanes/ethyl acetate to give 2.8 g (65%) of 9 as a white solid. NMR was consistent.
4-hydroxy-3-(3-(4-methoxyphenyl)-2-methyl-3-oxopropanoyl)phenyl 4-methoxybenzoate (10): To a suspension of 84 mg (2.1 mmol) of sodium hydride (60% dispersion in mineral oil) in 10 mL of anhydrous DMF at 0-5° C. under nitrogen was added a solution of 0.87 g (2 mmol) of 2-propionyl-1,4-phenylene bis(4-methoxybenzoate) in 2 mL of anhydrous DMF over 5 min. After 2 hours at 0-5° C., the reaction was quenched by the addition of 0.2 mL of glacial acetic acid. The reaction mixture was diluted with 20 mL of ethyl ether and saturated aqueous NaCl/H₂O (50 mL). The layers were separated, and the aqueous layer was extracted with 25 mL of ethyl ether. The combined organic layers were washed with 50:50 saturated aqueous NaCl/H₂O (30 mL), and organic dried over anhydrous Na₂SO₄, filtered, and concentrated in vacuo to a solid residue. The solid was crystallized from 100 mL of refluxing EtOAc under nitrogen by slow addition of 200 mL of hexanes and subsequent cooling to room temperature and then to 0-5° C. The solid was collected by filtration and dried 2 h under high vacuum at 60° C. to provide 0.43 g (50%) of intermediate 10. Compound 10 was used as such for the next deprotection step.
6-hydroxy-2-(4-methoxyphenyl)-3-methyl-4H-chromen-4-one (11): 4-hydroxy-3-(3-(4-methoxyphenyl)-2-methyl-3-oxopropanoyl)phenyl 4-methoxybenzoate (10, 0.43 g, 1 mmol) was suspended in 3.0 mL of acetic acid and 30 μL of sulfuric acid. After a 2.0 h reflux under nitrogen, the reaction was cooled to room temperature, diluted with 10 mL of hexanes, and filtered. The filter cake was washed with hexanes and then dried overnight under vacuum. The solid was purified by flash chromatography (hexanes/EtOAc, 1:1) to give 0.2 g (yield=71%) of 6-hydroxy-2-(4-methoxyphenyl)-3-methyl-4H-chromen-4-one (11) as a white solid. NMR was consistent.
6-hydroxy-2-(4-hydroxyphenyl)-3-methyl-4H-chromen-4-one (3): A Teflon-coated magnetic stir bar was added to an oven-dried, 100 mL round-bottomed flask. 6-hydroxy-2-(4-methoxyphenyl)-3-methyl-4H-chromen-4-one (11, 0.20 g, 0.71 mmol) in dry dichloromethane (40 mL) was added and the mixture cooled on an ice bath. After 20 min, boron tribromide (0.18 mL, 1.78 mmol, 2.5 equiv) was added at 0-5° C. and the reaction mixture was stirred at the same temperature for another 60 mins. Then, the reaction mixture was warmed to room temperature and stirred at room temperature for 18 h. Reaction progress was monitored by TLC. After completion, saturated bicarbonate solution (50 mL) was added slowly, and the solution was extracted with DCM (50 mL×2). The organic layers were combined and dried over anhydrous Na₂SO₄, filtered, evaporated, and chromatographed over silica gel eluting with Hexane: ethylacetate (1:4) to give 6-hydroxy-2-(4-hydroxyphenyl)-3-methyl-4H-chromen-4-one (3, 100 mg, 37%). NMR and LC/MS were consistent.

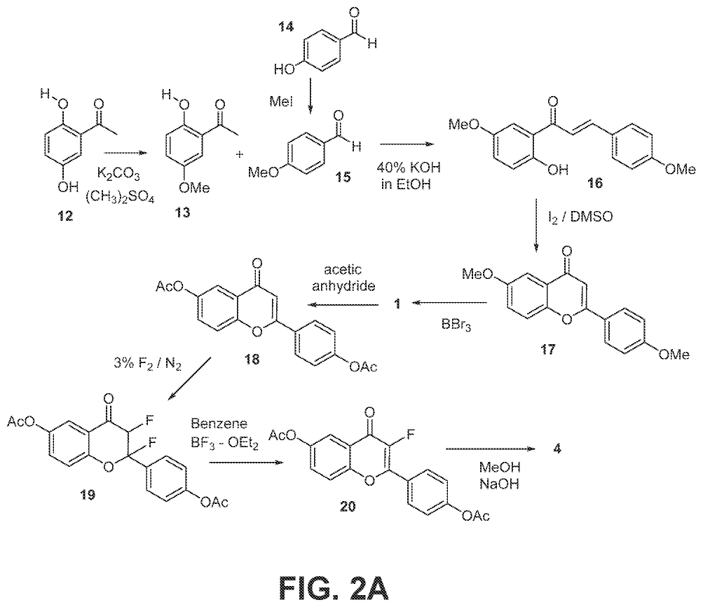

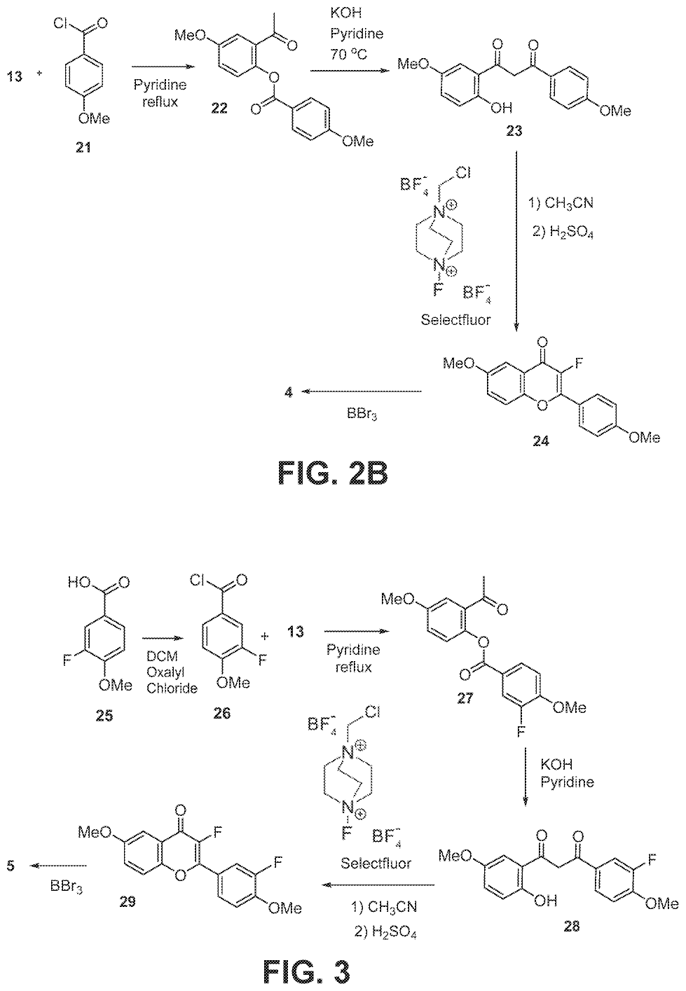

B. Synthesis of 6-hydroxy-2-(4-hydroxyphenyl)-4H-chromen-4-one (compound 1 of Table 1) and 3-fluoro-6-hydroxy-2-(4-hydroxyphenyl)-4H-chromen-4-one (compound 4 of Table 1). Following the synthesis scheme depicted in FIG. 2A:

1-(2-hydroxy-5-methoxyphenyl)ethanone (13); To a refluxed solution of compound 1-(2,5-dihydroxyphenyl)ethanone (12, 1.5 g, 9.87 mmol) and potassium carbonate (2.7 g, 19.74 mmol) in 100 mL anhydrous acetone, (CH₃)₂SO₄ (1.13 mL, 11.84 mmol) was added in drop wise. The reaction mixture was refluxed for 2 h. Progress of reaction was monitored by TLC. After 2 h, TLC showed no remaining starting material. The orange solution was diluted with dichloromethane (200 mL) and poured into cold water. The aqueous phase was extracted with dichloromethane and the combined organic extracts were washed with water, dried over anhydrous Na₂SO₄, filtered, and evaporated to yield crude 1-(2-hydroxy-5-methoxyphenyl) ethanone, which was purified by silica gel column chromatography (EtOAc:Hexanes=1:9) to afford 1.2 g (74%) methylated product 13. NMR data was consistent.
4-methoxybenzaldehyde (15); Suspension of (4.5 g 4 eq., 81.8 mmol) KOH in 100 mL of DMSO was stirred for 20 min before 4-hydroxy-benzaldehyde (14; 2.5 g, 20.5 mmol) and iodomethane (2 eq, 2.55 mL) were added. Stirring at room temperature was continued until TLC showed no remaining starting material. The orange solution was diluted with dichloromethane (300 mL) and poured into ice-water. The aqueous phase was extracted with dichloromethane and the combined organic extracts were washed with water, dried over anhydrous Na₂SO₄, filtered, and evaporated to yield crude 4-methoxybenzaldehyde (15), which was used in next step without further purification. Yield 2.7 g. NMR was consistent.
(E)-1-(2-hydroxy-5-methoxyphenyl)-3-(4-methoxyphenyl) prop-2-en-1-one (16); To a solution of 1-(2-hydroxy-5-methoxyphenyl)ethanone (13; 1.66 g, 10.0 mmol) in 12 mL of 40% KOH in ethanol, 4-methoxybenzaldehyde (15; 1.43 g, 10.5 mmol) was added and the mixture was stirred for 48 h at room temperature. The colored solution was poured into crushed ice and acidified with 1.0 N HCl (50 mL). The precipitate obtained was filtered and washed with cold water, dried, and recrystallized with absolute ethanol to get 16 (2.0 g, 70%). NMR data was consistent.
6-methoxy-2-(4-methoxyphenyl)-4H-chromen-4-one (17); To a solution of 5.28 mmol (1.50 g) of chalcone 16 in a 30 mL of DMSO taken in a 100 ml round bottom flask, fitted with reflux condenser, and a catalytic amount of iodine (50 mg) was added. The reaction mixture was refluxed for 4 h and allowed to cool to RT overnight. The precipitate was neutralized with sodium thiosulphate, filtered, washed with water, dried, and purified by silica gel column to afford 1.3 g (88%) white solid of desired flavone 17. NMR data was consistent.
6-hydroxy-2-(4-hydroxyphenyl)-4H-chromen-4-one (1); Compound 17 (6-methoxy-2-(4-methoxyphenyl)-4H-chromen-4-one, 0.50 g, 1.77 mmol) in 50 ml dry dichloromethane) was added to an oven-dried, 100 mL round-bottomed flask equipped with a Teflon-coated magnetic stir bar. The mixture was then cooled on an ice bath. After 10 min, boron tribromide (0.67 mL, 7.08 mmol) was added at 0° C. and the reaction mixture was stirred at the same temperature for another 30 mins. Then, the reaction mixture was stirred at room temperature for 18 h. Reaction progress was monitored by TLC. After completion, water (50 mL) was added carefully, and the solution was extracted with DCM (100 mL). The organic layer was dried over anhydrous Na₂SO₄, filtered, and evaporated. The organic phase was purified by column chromatography (Hexanes:Ethylacetate 20:80) to afford 300 mg (67%) of 1. NMR data was consistent.
4-(6-acetoxy-4-oxo-4H-chromen-2-yl)phenyl acetate (18); 6-hydroxy-2-(4-hydroxyphenyl)-4H-chromen-4-one (1, 254 mg, 1.0 mmol) was dissolved in acetic anhydride (5 mL) and pyridine (5 mL), and the solution was stirred at room temperature for 2 h. After completion, the reaction mixture was poured into ice-water (10 mL), and the precipitate was collected by filtration and purified by flash chromatography on a column of silica gel eluted with CH₂Cl₂/MeOH (20:1) to yield compound 18 (200 mg, 59%) as a white powder. NMR data was consistent.
4-(6-acetoxy-3-fluoro-4-oxo-4H-chromen-2-yl)phenyl acetate (20); Fluorine, at concentrations of 3% in N₂, was slowly passed through a cold (−50° C.) and vigorously stirred solution of the flavone 18 (100 mg, 0.30 mmol) dissolved in 25 mL of CFCl₃, 30 mL of CHCl₃, and 6 mL of EtOH. An efficient mixing was achieved by using a magnetic stirrer, which also ensured a fine dispersion of the gas bubbles. Reaction progress was monitored by TLC. The reaction was quenched by pouring into 200 mL water, washing the organic layer with NaHCO₃ solution followed by water until neutral, drying the organic layer over anhydrous MgSO₄, filtered and finally evaporating the solvent. The crude product (19) was purified by flash chromatography using silica gel with various proportions of Hexanes/EtOAc as eluent. Purified product was used as such for the next dehydro fluorination step. Difluoro derivative 19 (60 mg) was dissolved in dry benzene (5 mL) and cooled to about 10° C. Excess BF₃·OEt₂ (3 mL) was added in one portion, and the reaction mixture allowed to warm to room temperature and stirred for an additional 3 h. Cold diluted 1N HCl solution (10 mL) was added, and the organic layer washed with bicarbonate and dried over anhydrous sodium sulphate, filtered and concentrated to give 20. Crude was used as such for next final deprotection step.
3-fluoro-6-hydroxy-2-(4-hydroxyphenyl)-4H-chromen-4-one (4); To a stirring mixture of 4-(6-acetoxy-3-fluoro-4-oxo-4H-chromen-2-yl)phenyl acetate (20, 71 mg, 0.20 mmol) in methanol (10 mL) was added a solution of sodium hydroxide (0.5 M, 1.0 mL) at 0° C. under N₂ atmosphere. After completion, 100 mL ethyl acetate was added to the reaction mixture. The ethyl acetate layer was washed with water (100 mL), and then with brine (100 mL). The organic layer was dried over anhydrous Na₂SO₄. The crude material was filtered, concentrated, and purified by silica gel column chromatography (100% ethyl acetate) to afford the desired 3-fluoro-6-hydroxy-2-(4-hydroxyphenyl)-4H-chromen-4-one (4, 40 mg, 87%). NMR and LC/MS data were consistent.

C. Alternative synthesis of 3-fluoro-6-hydroxy-2-(4-hydroxyphenyl)-4H-chromen-4-one (compound 4 of Table 1). Following the synthesis scheme depicted in FIG. 2B:

2-acetyl-4-methoxyphenyl 4-methoxybenzoate (22); A dry round-bottomed flask equipped with a reflux condenser charged with 1-(2-hydroxy-5-methoxyphenyl)ethanone (13, 3.32 g, 20.0 mmol), p-methoxybenzoyl chloride (21; 4.09 g, 24.0 mmol, 1.2 equiv), and dry pyridine (40.0 mL) was heated at 100° C. for 4 h. The mixture was then poured into a mixture of ice and 1.0 N hydrochloric acid (70 mL) and extracted with ethyl acetate. The extract was washed with water and aqueous sodium carbonate (50 mL) and dried over anhydrous sodium sulfate. The material was filtered and the solvent was then evaporated under reduced pressure to give the crude product. Crude 22 was used as such for next step.
1-(2-hydroxy-5-methoxyphenyl)-3-(4-methoxyphenyl)propane-1,3-dione (23): To a solution of the crude product 22 in pyridine (30.0 mL) was added freshly powdered potassium hydroxide (5.3 g), and the resultant mixture was vigorously stirred at 70° C. for overnight and then poured into a mixture of ice and 1.0 N hydrochloric acid (100 mL) and extracted with ethyl acetate. The extract was washed with 1.0 N hydrochloric acid (100 mL) and aqueous sodium carbonate (200 mL), and the organic solvent dried over anhydrous Na₂SO₄, filtered and evaporated via rotary evaporation. The residue was recrystallized from methanol to afford yellow crystals (4.7 g, 78%). Crude was purified through Silica gel column chromatography (eluent=DCM). NMR was consistent.
3-fluoro-6-methoxy-2-(4-methoxyphenyl)-4H-chromen-4-one (24); The mixture of 1-(2-hydroxy-5-methoxyphenyl)-3-(4-methoxyphenyl)propane-1,3-dione (23, 1.36 g, 4.52 mmol, 1 equivalent) and Selectfluor (1.92 g, 5.43 mmol, 1.2 equivalents) was stirred with anhydrous CH₃CN (4.0 mL) for 15 h at ambient temperature. Concentrated sulfuric acid (450 μL) was then added dropwise to the mixture and the resulting mixture was continuously stirred for 0.5 h at room temperature. The mixture was poured into water and neutralized with NaOH (10%, 9 mL). The solution was extracted with dichloromethane (40 mL×3). The organic layer was combined and dried over Na₂SO₄. The material was filtered and the volatiles were removed under the reduced pressure and the oil residue was purified by column chromatography (Hexanes/ethyl acetate 90:10) to afford the desired product 24 as a white solid (1.0 g, 74% yield). NMR was consistent.
3-fluoro-6-hydroxy-2-(4-hydroxyphenyl)-4H-chromen-4-one (4); Boron tribromide (1.89 mL, 20.0 mmol, 6 equivalents) was added to a stirred solution of 3-fluoro-6-methoxy-2-(4-methoxyphenyl)-4H-chromen-4-one (24, 1.0 g, 3.33 mmol) in dry CH₂Cl₂ (100 mL) under N₂ at −10° C. Upon complete addition of BBr₃, the reaction was maintained at −10° C. for 30 min and then allowed to reach room temperature and stir for an additional 16 h. The mixture was cooled to 0° C. and carefully quenched with H₂O (30 mL). The resulting precipitate was filtered and washed with water (100 mL). Drying under high vacuum gave final compound 4 (600 mg, 66% yield). NMR was consistent.

D. Synthesis of 3-fluoro-6-hydroxy-2-(3-fluoro-4-hydroxyphenyl)-4H-chromen-4-one (compound 5 of Table 1). Following the synthesis scheme depicted in FIG. 3:

3-Fluoro-4-methoxybenzoyl chloride (26): Compound 25 (3-fluoro-4-methoxy-benzoic acid, 2.05 g, 12.1 mmol) was added to a dry round bottom flask with a stir-bar. Oxalyl chloride (1.5 mL) in dichloromethane (DCM/Methylene chloride; 10 mL) was then added. In addition, anhydrous dimethylformamide (DMF; 200 μL) was added to the reaction which was stirred at room temperature (2 hrs). Solvent was removed via reduced pressure to afford an off-white solid 26 which was used in the next step.
3-acetyl-5-methoxyphenyl 3-fluoro-4-methoxybenzoate (27): To crude (26) in the same round bottom flask containing a stir bar, dry pyridine (30 mL) was added. To the stirring solution, 1-(2-hydroxy-5-methoxyphenyl) ethanone (13; 1.82 g; 10.9 mmol). A reflux condenser was attached to the round bottom flask and the mixture refluxed (100° C.; 4 hrs). The reaction mixture was then allowed to cool, and the solution was poured over ice cold 1.0 M HCl (100 mL). The resulting solution was extracted with ethyl acetate (100 mL). The organic extract was washed with water and saturated aqueous sodium carbonate (50 mL). The organic phase was dried over anhydrous sodium sulfate, filtered, and solvent was removed via rotary evaporation to afford crude 27. NMR was consistent.
1-(3-fluoro-4-methoxyphenyl)-3-(2-hydroxy-5-methoxyphenyl) propane (28): In a dry round bottom flask, crude (27), dry pyridine (35 mL), and a stir bar were added. To the stirring solution, finely crushed KOH (5.04 g) was added. The round bottom flask was fitted with a condenser and refluxed (70° C. 4 hrs). After refluxing, the solution was cooled with constant stirring. A solution of 20% glacial acetic acid (200 mL) was added. Acidified solution was then stirred (3 hrs). The resulting product was filtered and dried via Buchner filtration and washed with cold deionized water. Resulting product (28) afforded an orange powder (1.32 g; 4.56 mmol). NMR was consistent.
3-fluoro-2-(3-fluoro-4-methoxyphenyl)-6-methoxy-4H-chromen-4-one (29): To a dry round bottom flask crude (28) and acetonitrile (15.0 mL), and a small stir bar were added. To a stirring solution, Selectfluor (1.93 g; 5.45 mmol) was added and stirred at RT (17 hrs). Concentrated sulfuric acid (300 μL) was then added dropwise to the stirring solution and stirred for an additional (0.5 hr). The solution was extracted by adding deionized water (50 mL) and extracting with DCM (3×30 mL). Organic layer was dried with anhydrous sodium sulfate, filtered, and solvent removed via reduced pressure to afford 1.37 g of crude orange oily product. A dry column was performed utilizing a Hexanes: Dichloromethane mixture and assisted via pressure of a vacuum. Pure product was recovered (230 mg; 0.72 mmol). NMR was consistent.
3-fluoro-2-(3-fluoro-4-hydroxyphenyl)-6-hydroxy-4H-chromen-4-one (5): Compound 29 (230 mg, 0.72 mmol) was added to a dry round bottom flask with a stir bar under an inert atmosphere. Dry dichloromethane (15.0 mL) was added and cooled (−10° C.). Boron tribromide (6 equivalents; 0.4 mL) was added to the stirring solution. Solution was allowed to warm to ambient temperature and stirred (12 hrs). After stirring, solution was cooled (0° C.) and quenched with deionized water (10 mL). Precipitate was filtered via Buchner funnel filtration and washed with cold deionized water (50 mL). Precipitate was allowed to dry under vacuum conditions. Product afforded (5; 66 mg) as brown crystals. NMR was consistent.

### Example 2

### In Vitro Testing of Flavone Compounds 3 and 4 of this Disclosure

A screen for the agonist or antagonist effects of a set of commercially available flavonoids was done by assessing their ability to induce or inhibit steroid hormone receptor-mediated gene expression. Cell lines were used in which the activity of endogenous genes or stably integrated reporter genes provided a facile method to quantify the effect of the flavonoids. The compound library was tested for their effects on gene expression mediated by the estrogen receptor, androgen receptor, progesterone receptor, and glucocorticoid receptor. Our data confirmed published data that compound 1 (6-hydroxy-2-(4-hydroxyphenyl)-4H-chromen-4-one) was an estrogen agonist. We also found that compound 2 (3,6-dihydroxy-2-(4-hydroxyphenyl)-4H-chromen-4-one) exhibited superior potency as an estrogen receptor agonist compared to compound 1. Compound 2 differs from compound 1 by the addition of a hydroxyl group at carbon 3 of the flavone scaffold. We reasoned that other substitutions at position 3 could have estrogenic activity. Two novel compounds were tested: compound 3 (6-hydroxy-2-(4-hydroxyphenyl)-3-methyl-4H-chromen-4-one) and compound 4 (3-fluoro-6-hydroxy-2-(4-hydroxyphenyl)-4H-chromen-4-one) of Table 1, which have a methyl or fluoro moiety, respectively, at position 3.

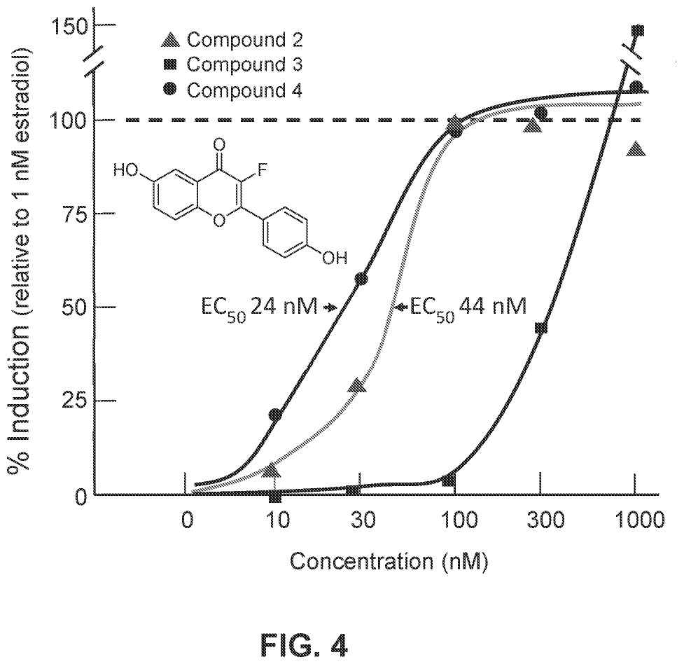

Results from cell culture-reporter gene assay was performed in which a T47D breast cancer cell line with a stably integrated Estrogen Response Element (ERE)-luciferase reporter gene was treated for 20 hours with the indicated doses of test compounds (FIG. 4). Luciferase activity was compared to an optimal dose of estradiol (1 nM).

In this reporter gene assay, compound 3 of Table 1 was about 10-fold less potent than compound 2, although at very high doses appears to have superagonist activity. In contrast, the potency of compound 4 had robust estrogenic activity and superior potency compared to compound 2, EC₅₀ (24 nM versus 44 nM, as shown in FIG. 4).

### Example 3

### Regeneration of the Vaginal Epithelium by Compound 4 of this Disclosure

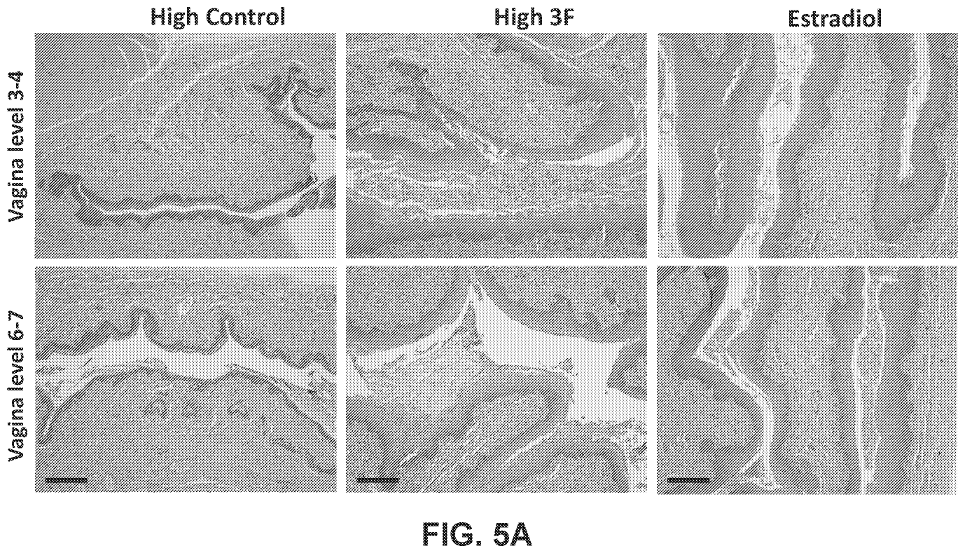

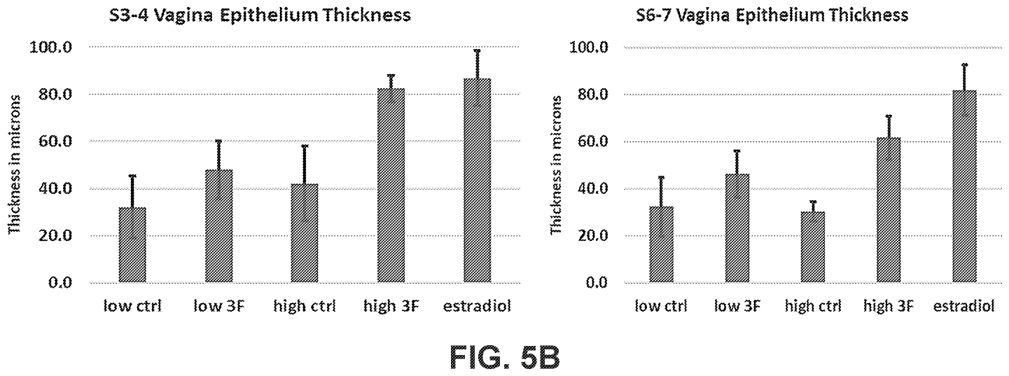

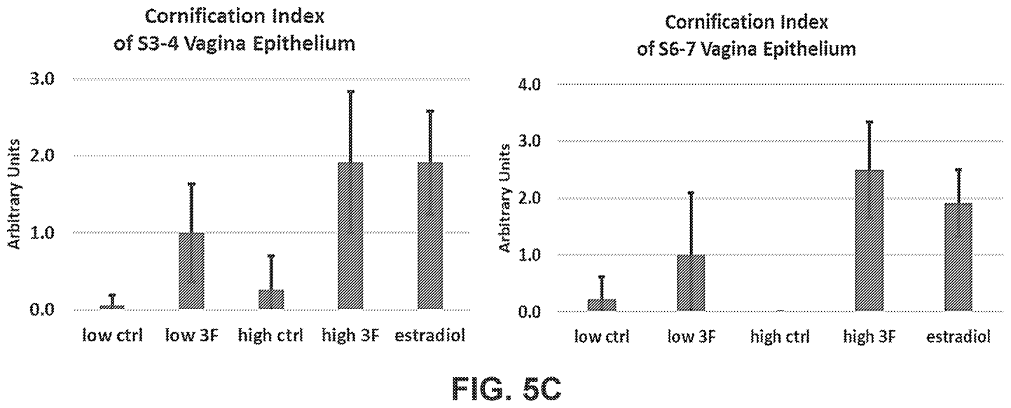

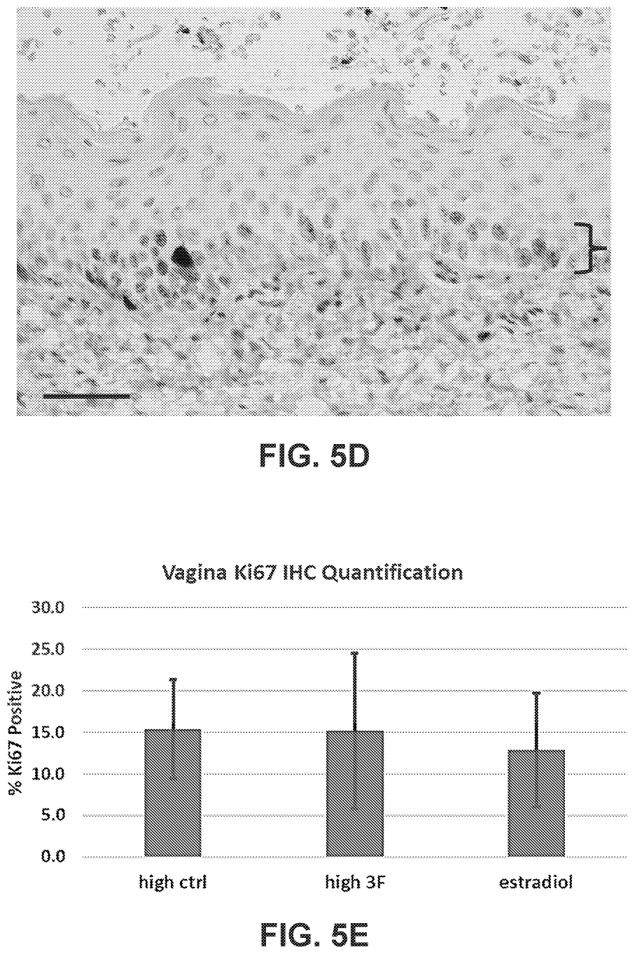

Compound 4 of Table 1 was tested in the ovariectomized rat, a model of GSM, (Yousefzadeh N, et al., Ovariectomized rat model of osteoporosis: a practical guide. EXCLI J. 2020 Jan. 10; 19:89-107. doi: 10.17179/excli2019-1990). Six rats were tested in each of five treatment groups: rats treated with low or high concentrations of control (vehicle only); rats treated with low (0.2 mg/suppository or high (1.0 mg/suppository levels of compound 4 (abbreviated as “3F” in these figures); and rats treated with estradiol 0.1 microgram/suppository. Suppositories were administered intravaginally daily for 14 days. There were two sets of vehicle controls, corresponding to the level of excipients used in the in the low and high compound 4 treatment conditions. The estradiol treatment condition had excipients equivalent to the high control and high compound 4 treatment conditions. The individuals performing the drug treatment of the animals, their gross evaluations, sacrifice and dissection were blinded to the treatment that each group received. FIG. 5A shows representative histological images (hematoxylin and eosin staining) from vaginal levels 3-4 (mid-vagina) and 6-7 (near cervix), as described by Berger et al. (Berger L, El-Alfy M, Martel C, Labrie F. Effects of dehydroepiandrosterone, Premarin and Acolbifen on histomorphology and sex steroid receptors in the rat vagina. J. Steroid Biochem Mol Biol. 2005 July; 96(2):201-15. doi: 10.1016/j.jsbmb.2005.02.018.PMID: 15979306 and Berger L, El-Alfy M, Labrie F. Effects of intravaginal dehydroepiandrosterone on vaginal histomorphology, sex steroid receptor expression and cell proliferation in the rat. J Steroid Biochem Mol Biol. 2008 March; 109(1-2):67-80 doi: 10.1016/j.jsbmb.2007.09.023). In these images, the size bar equals 200 microns. To determine the depth of the epithelium, high quality 100x images were taken of the two regions and thickness of the epithelium measured at 25 distinct sites at each level for each animal, then averaged. The values obtained for the six animals in each group were averaged to obtain the mean epithelial depth for that group as indicated by the bar graphs of FIG. 5B (in these bar graphs, vertical lines represent plus or minus one standard deviation of the mean of the epithelial thickness). P values were determined by student's T-test. Both the high compound 4 and estradiol treatment were significantly different than the corresponding control (p<0.001). The effect of compound 4 was dose dependent. The low compound 4 treatment was borderline significant in both regions (p≈0.05). The evaluator was blinded to the treatment of the groups. These experiments show that compound 4 can restore the structure of the degenerate vaginal epithelium to the same level as estradiol.

To evaluate the ability of compound 4 to also restore proper function of the vaginal epithelium several parameters were examined. The lining of the rat vagina is keratinized. Keratinization of the vaginal epithelium is regulated by estrogen signaling. A relative cornification index was established with values between 0 (none) and 3 (very thick) at increments of 0.5 and a relative cornification index was arrived at for each vagina. The mean plus/minus standard deviation for vaginal sections 3-4 and 6-7 are depicted by the bars and vertical lines, respectively, of FIG. 5C and p values were determined by student's T-test. Both high compound 4 treatment and estradiol treatment are significantly different than the control p≤0.01. The low compound 4 treatment was borderline significant in both regions 3-4 and 6-7 (p≤0.05).

Ki-67 is a protein expressed in G1, S, G2, and M phase and has been established as a marker of cellular proliferation. Ki67 immunohistochemistry (IHC) was performed on a vaginal section from animals in the control, the high concentration compound 4, and the estradiol-treated groups. A representative image of the Ki67 positive cells (dark staining) is shown in FIG. 5D. Only the basal and parabasal layers were considered (bracket), size bar equals 200 microns. Approximately 800 cells were assessed on each slide. The percent Ki67 positive cells for the six animals in a group were averaged, and the mean value is depicted graphically in FIG. 5E by bars for each of the three groups with vertical lines indicating plus/minus one standard deviation. P values were determined by student's T-test. There was no statistical difference between the groups. These results indicate that estradiol and compound 4 do not increase the depth of the vaginal epithelium by stimulating cell proliferation but, rather, by promoting survival and differentiation of the squamous epithelium.

### Example 4

### Evaluation of Uterine Endometrium, Myometrium, and Bladder Urothelium Growth Following Treatment with Compound 4

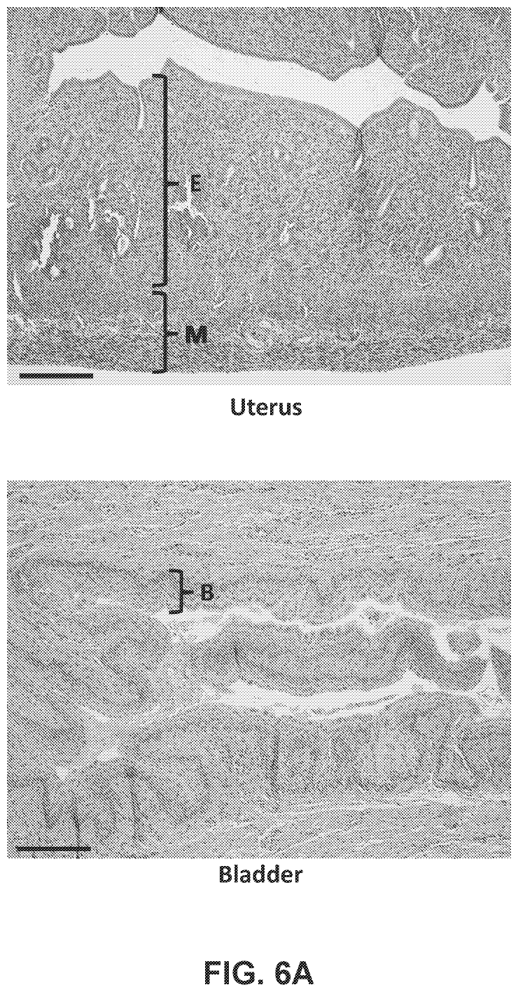

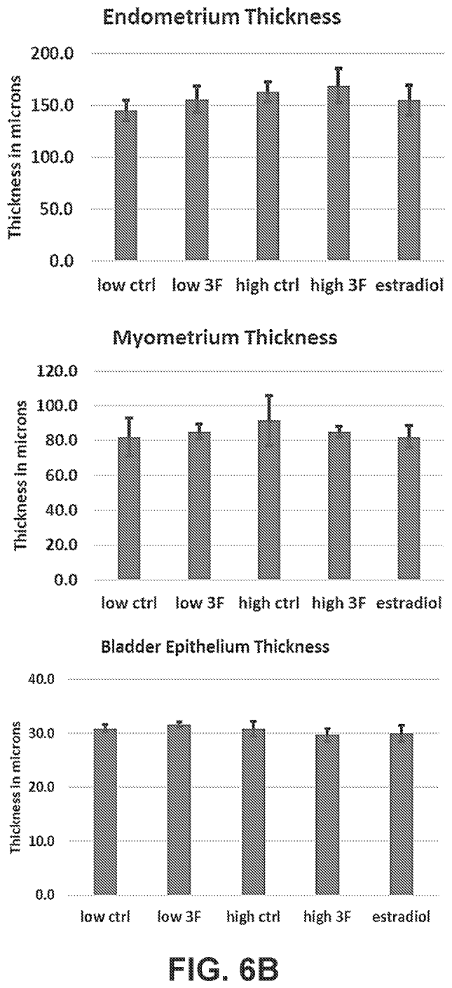

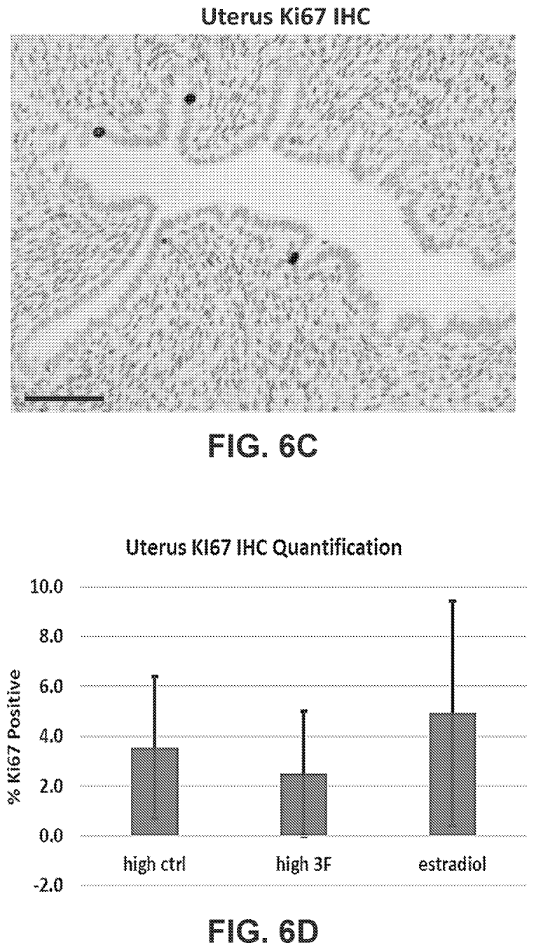

To ascertain whether compound 4 has an effect on the epithelium of anatomically neighboring tissues, the thickness of the epithelium was assessed in the uterus (a known target tissue of physiological levels of estradiol) and bladder (not an estrogen target tissue). The rats from the five treatment groups in the ovariectomized rat model of GSM described in Example 2 were evaluated for the effects of compound 4 on growth of uterus and bladder. FIG. 6A shows a representative 40x image from the uterus with designations of the endometrium (bracket E) and myometrium (bracket M) (size bar equals 500 microns). A representative bladder image is also shown and the urothelium indicated (size bar equals 200 microns). The endometrium, myometrium, and urothelium thickness of each animal (n=6) were measured in 20 locations and averaged. Those averages were used to obtain the group mean. The mean of each treatment group is depicted in the bar graphs of FIG. 6B. Standard deviation is represented as a vertical line. P values were determined by student T-test. None of the pairs exhibited a significant difference.

Ki67 IHC was performed on a uterine section from animals in the control, the high level of compound 4 (abbreviated as “3F” in these figures), and estradiol groups. A representative image of the Ki67 positive cells (dark stain) is shown in FIG. 6C (size bar equals 200 microns). Approximately 800 cells were assessed for each animal within a group, then averaged to obtain the group mean. The mean percent Ki67 positive cells of each of the three treatment groups is shown in the bar graph in FIG. 6D. Standard deviation for each group, n=6, is represented as a vertical line. P values were determined by student T-test. None of the pairs exhibited a significant difference.

These data demonstrate that compound 4 does not stimulate the growth of uterine endometrium, myometrium, or bladder urothelium.

### Example 5

### Compound 4 Stimulates Vaginal Lactoferrin Production

Lactoferrin is an iron-binding protein that, as part of the innate immune system, has anti-microbial properties due to its ability to sequester iron. Lactoferrin gene expression is stimulated by estrogens in the vagina. The ability of vaginally administered compound 4 to recapitulate this beneficial action of estrogens was assessed by immunohistochemical analysis of lactoferrin in the vaginas of rats from the experiment described in example 2.

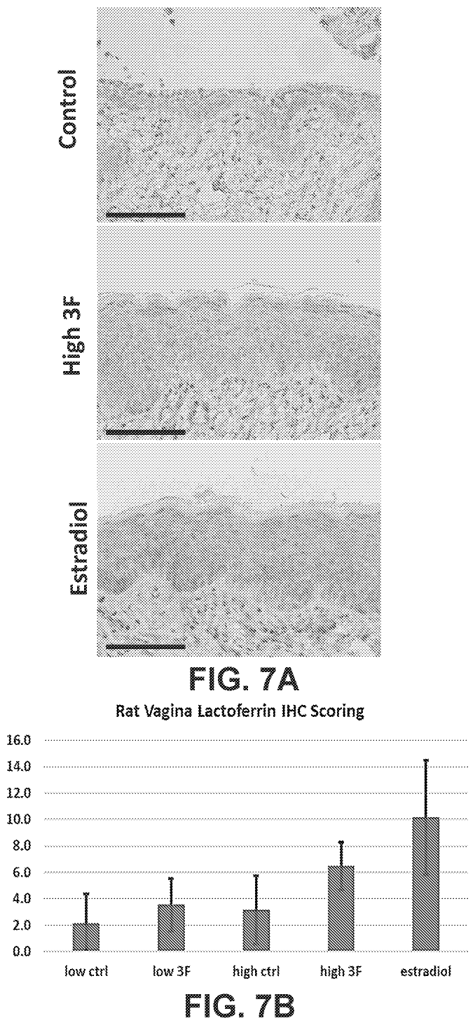

FIG. 7 shows representative slides from vaginas treated with high compound 4, estradiol, and high vehicle (control) and stained for lactoferrin. A semi-quantitative analysis was devised and the results are depicted graphically (FIG. 7B). The high compound 4-treated animals exhibited higher lactoferrin expression than controls, p=0.027, but their lactoferrin expression was not significantly distinguishable from estradiol-treated animals, p=0.084

### Example 6

### Compound 4 Stimulates Vaginal Mucous Production

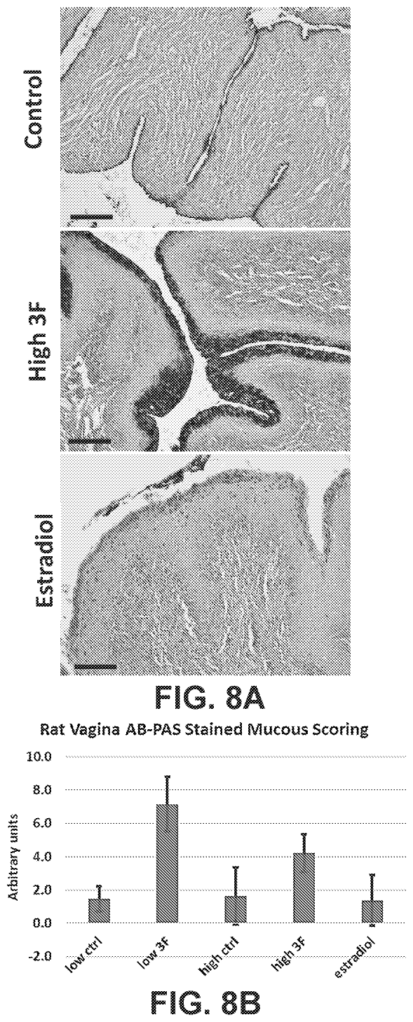

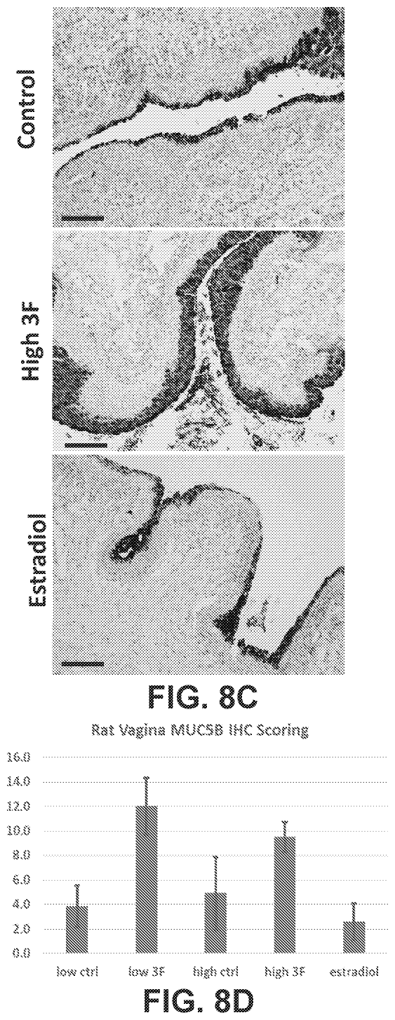

Mucins play an important protective role in the vagina as anti-microbials and as a lubricant. The rats from the five treatment groups in the ovariectomized rat model of GSM described in Example 2 were evaluated for the effects of compound 4 on vaginal mucous production. Representative Alcian Blue-Periodic acid-Schiff (AB-PAS) stained images from the vaginal sections of animals in the high control, the high dose of compound 4 (abbreviated as “3F” in these figures), and estradiol groups are shown in FIG. 8A (size bar equals 100 microns).

A semi-quantitative scoring system for the mucous was designed with values between 0 to 2 in increments of 0.5 for the quantity of mucous and the depth of the epithelium positive for mucous. Assessment was carried out independently for vaginal levels 1, 2&3, 4&5, and 6&7. The mucous in the vaginal lumen was also assessed, then all values were summed for each animal. The group mean is shown in the bar graph of FIG. 8B (vertical lines represent the standard deviation). P values were determined by student's T-test. Both the low compound 4 treatment and high compound 4 treatment were significantly different than the appropriate vehicle controls and estradiol treatment, p≤0.01.

To investigate compound 4-mediated production of mucins in more detail, we investigated the production and presence of mucin 5B in the rat vagina. Mucin 5B is an acidic mucin that is one of the major mucins of both rat and human genital tract. Immunohistochemistry specific for mucin 5B was performed on vaginal sections from each animal in the five treatment groups. Representative images from the mucin 5B immunohistochemistry staining of vaginas of animals in the control, the high compound 4, and estradiol groups are shown in FIG. 8C (size bar equals 100 microns). A semi-quantitative scoring system for the mucin 5B immunohistochemistry was designed with values between 0 to 2 in increments of 0.5 for the quantity of mucin 5B and the depth of the epithelium positive for mucin 5B, and then assessed at the levels 1, 2&3, 4&5, and 6&7. The presence of mucin 5B in the vaginal lumen was also assessed, then all values were summed for each animal. The group mean is shown in the bar graph in FIG. 8D (vertical lines represent the standard deviation). P values were determined by student's T-tests. Both the low compound 4 treatment and high compound 4 treatment were significantly different than the appropriate vehicle controls and estradiol treatment, p≤0.006. These data show that, in this animal model, compound 4, but not estradiol, stimulates the vaginal production of mucins. This, plus the observation that the lower dose of compound 4 treatment results in somewhat higher mucin production than higher dose of compound 4 suggests that compound 4 may be acting upon a pathway independent of the estrogen receptor and that this activity is beneficial toward reestablishing appropriate moisturization and barrier function of the vaginal epithelium.

The data obtained in these examples demonstrate that compound 4 restores the structure of vaginal epithelium and cellular functions, such as mucification, keratinization, and lactoferrin production, Animals from the experiment described in example 2 treated with compound 4 do not exhibit growth of the uterine endometrium nor stimulation of DNA replication in uterus or vagina. Compound 4 does not affect animal behavior, appearance, weight, or necropsy findings. Compound 4 was undetectable (LOD 0.33 ng/ml) in the systemic circulation one day after a two-week, daily suppository administration regimen.

### Example 7

### Compounds 4 and 5 Stimulate Growth of Estrogen-Dependent Cells

A difluorinated compound was synthesized (compound 5, Table 1) and its ability to stimulate growth of estrogen-dependent cells compared to compound 4. Estrogen-responsive MCF-7EV cells were plated in complete medium (Dulbecco's Modified Eagles Medium with high glucose, DMEM, supplemented with 10% fetal bovine serum, penicillin, and streptomycin) at 1000 cells per well in a 6 well dish for three days. The medium was removed and replaced with phenol red-free DMEM with 10% charcoal-stripped fetal bovine serum plus antibiotics. The cells were then treated with compound 4 or compound 5 (designated as 3F and 33′DiF in the figure, respectively) at the doses indicated. Additional wells were treated with vehicle alone or with estradiol (100 pM). Cells were allowed to grow for 10 days at which time medium was removed, the cells fixed in formalin, then stained with crystal violet. The stained cells were photographed, and the digital image processed using the Image J plugin to quantify the extent of plate coverage and the density of staining.

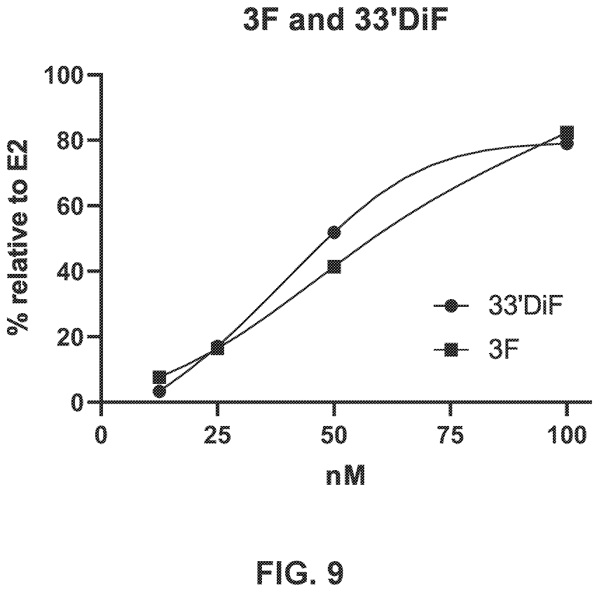

The growth index was calculated by adding the % plate coverage and the density readings and is shown graphically for compounds 4 and 5 in FIG. 9. The value for the estradiol-treated cells was set at 100. The EC₅₀ for estradiol is ≈2 pM, whereas the EC₅₀s of compounds 4 and 5 are about 50 nM. Therefore, the potency of compounds 4 and 5 are 1/25,000 that of estradiol in this sensitive assay for stimulation of estrogen-dependent growth of breast cancer cells.

The various features and methods described above may be used independently of one another or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

The present disclosure is not to be limited in scope by the specific embodiments described herein which are intended as single illustrations of individual aspects of this invention, and functionally equivalent methods and components are within the scope of this disclosure. Indeed, various modifications of this disclosure, in addition to those shown and described herein will become apparent to those skilled in the art from the foregoing description and accompanying drawings. Such modifications are intended to fall within the scope of the claims.

What is claimed is:

1. A flavone compound, or a pharmaceutically acceptable salt thereof, having the chemical structure: 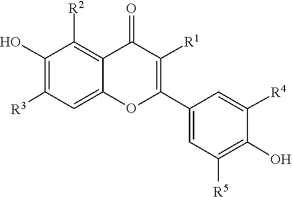
wherein: R¹ is methyl, ethyl, propyl, butyl, O-methyl, O-ethyl, O-propyl, O-butyl, methyl-OH, ethyl-OH, or halogen, and wherein R², R³, R⁴, and R⁵ are each independently hydrogen or halogen.

2. A flavone compound of claim 1, or a pharmaceutically acceptable salt thereof, having a chemical structure selected from the group consisting of:

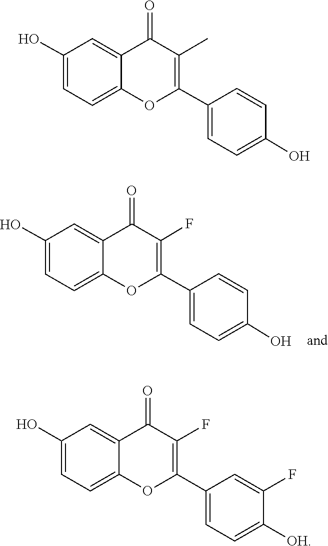 [SMILES: Cc1c(-c2ccc(O)cc2)oc2ccc(O)cc2c1=O ; O=c1c(F)c(-c2ccc(O)cc2)oc2ccc(O)cc12 ; O=c1c(F)c(-c2ccc(O)c(F)c2)oc2ccc(O)cc12]

3. A pharmaceutical composition comprising a compound of claim 1 or 2, and at least one pharmaceutically acceptable carrier.

4. The pharmaceutical composition of claim 3, wherein the composition is formulated for intravaginal administration.

5. The pharmaceutical composition of claim 3, wherein the composition is formulated as a lotion.

6. The pharmaceutical composition of claim 3, wherein the composition is formulated as a cream.

7. The pharmaceutical composition of claim 3, wherein the composition is formulated as a vaginal ring.

8. A pharmaceutical kit containing a pharmaceutical composition of any one of claims 3-7, prescribing information for the composition, and a container.

9. A method of treating genitourinary syndrome of menopause in an individual comprising administering a therapeutically effective amount of a compound of claim 1 or 2, or a pharmaceutical composition of any one of claims 3-7, to an individual in need thereof.

10. The method of claim 9, wherein symptoms of the genitourinary syndrome of menopause comprise at least one of vulvovaginal atrophy, vaginal dryness, prurutis, incontinence, dyspareunia, and recurrent urinary tract infections.

11. The method of claim 9, wherein the administering ameliorates at least one of vulvovaginal atrophy, vaginal dryness, incontinence, dyspareunia, and recurrent urinary tract infections in the individual.

12. The method of claim 9, wherein the individual has vulvovaginal atrophy resulting from loss of estrogen during perimenopause or menopause.

13. The method of claim 9, wherein the individual has vulvovaginal atrophy resulting from surgical removal of ovaries.

14. The method of claim 9, wherein the individual has vulvovaginal atrophy resulting from use of chemically induced estrogen suppression during treatment of breast cancer.

15. The method of claim 9, wherein the compound or composition is administered in an amount sufficient to cause at least one of increased depth of the vaginal epithelium, increased mucin production in the individual's vagina, increased keratin fiber synthesis and keratin fiber cross-linking in the individual's vagina, and increased vascularization and blood flow in the individual's vagina.

16. The method of claim 9, wherein the individual is a human.

17. A compound of claim 1 or 2, or a pharmaceutical composition of any one of claims 3-7, for use in the treatment of genitourinary syndrome of menopause in an individual.

18. The use of claim 17, wherein symptoms of the genitourinary syndrome of menopause comprise at least one of: vulvovaginal atrophy, vaginal dryness, prurutis, incontinence, dyspareunia, and recurrent urinary tract infections.

19. The use of claim 17, wherein the use ameliorates at least one of vulvovaginal atrophy, vaginal dryness, incontinence, dyspareunia, and recurrent urinary tract infections in the individual.

20. The use of claim 17, wherein the individual has vulvovaginal atrophy resulting from loss of estrogen during perimenopause and/or menopause.

21. The use of claim 17, wherein the individual has vulvovaginal atrophy resulting from surgical removal of ovaries.

22. The use of claim 17, wherein the individual has vulvovaginal atrophy resulting from use of chemically induced estrogen suppression during treatment of breast cancer.

23. The use of claim 17, wherein the use results in at least one of increased depth of the vaginal epithelium, increased mucin production in the individual's vagina, increased keratin fiber synthesis and keratin fiber cross-linking in the individual's vagina, and increased vascularization and blood flow in the individual's vagina.

24. Use of a compound of claim 1 or 2, or a pharmaceutical composition of any one of claims 3-7, in the manufacture of a medicament for the treatment of genitourinary syndrome of menopause in an individual.

25. The use of claim 24, wherein symptoms of the genitourinary syndrome of menopause comprise at least one of vulvovaginal atrophy, vaginal dryness, prurutis, incontinence, dyspareunia, and recurrent urinary tract infections.

26. The use of claim 24, wherein the use ameliorates at least one of: vulvovaginal atrophy, vaginal dryness, incontinence, dyspareunia, and recurrent urinary tract infections in the individual.

27. The use of claim 24, wherein the individual has vulvovaginal atrophy resulting from loss of estrogen during perimenopause and/or menopause.

28. The use of claim 24, wherein the individual has vulvovaginal atrophy resulting from surgical removal of ovaries.

29. The use of claim 24, wherein the individual has vulvovaginal atrophy resulting from use of chemically induced estrogen suppression during treatment of breast cancer.

30. The use of claim 24, wherein the use results in at least one of increased depth of the vaginal epithelium, increased mucin production in the individual's vagina, increased keratin fiber synthesis and keratin fiber cross-linking in the individual's vagina, and increased vascularization and blood flow in the individual's vagina.
